(19) United States
(12) Patent Application Publication
(10) Pub. No.: US 2023/0088574 A1
(43) Pub. Date: Mar. 23, 2023

(54) SYSTEM FOR DRAWING FLUID FROM A BAG UNDER SUB-AMBIENT CONDITIONS

(71) Applicant: MODULAR MEDICAL, INC., San Diego, CA (US)

(72) Inventors: Paul M. DiPerna, Escondido, CA (US); Marc D. Goldman, San Diego, CA (US); David Neese, Escondido, CA (US); Patrick Hearty, Vista, CA (US)

(21) Appl. No.: 17/591,435

(22) Filed: Feb. 2, 2022

Related U.S. Application Data
(63) Continuation-in-part of application No. 17/482,848, filed on Sep. 23, 2021.

(51) Int. Cl. A61M 5/152; A61M 5/168

(52) U.S. Cl. CPC A61M 5/152; A61M 5/16881; A61M 2205/3331; A61M 2005/16868; A61M 2005/16872

(57) ABSTRACT

A portable system and method for infusing liquid medicaments to a patient through an elastomeric fluid channel includes an airtight pressure shell for holding a collapsible bag of the fluid medicament to be infused. A pressure sensor connected to the pressure shell monitors decreases below ambient pressure inside the shell to determine when the sub-ambient pressure becomes insufficient to continue assisting the withdrawal of fluid from the collapsible bag to an external pinch/squeeze unit. An equilibration valve is synchronized with the pinch/squeeze unit to reestablish ambient pressure in the pressure shell for continued operation as the pinch/squeeze unit acts to sequentially push liquid medicament for infusion into the patient.

### REFERENCE TO RELATED APPLICATIONS

This application is a continuation-in-part of application Ser. No. 17/482,848, filed Sep. 23, 2021, which is currently pending. The contents of application Ser. No. 17/482,848 are incorporated herein by reference.

### FIELD OF THE INVENTION

The present invention pertains to systems and methods for infusing fluid medicaments from a collapsible bag to a patient. More particularly, the present invention pertains to portable pumps that establish and create infusion pressures on the collapsible bag which are below ambient pressure. The present invention is particularly, but not exclusively, useful as an infusion system wherein motive forces interact radially with an elastomeric fluid channel carrying the fluid medicament. Wherein this interaction is controlled by monitoring decreases below ambient air pressure acting on a collapsible bag containing the fluid medicament, and equilibrating the pressure shell to ambient pressure when an overpressure is insufficient to infuse fluid medicament to the patient in accordance with a predetermined protocol.

### BACKGROUND OF THE INVENTION

Insulin infusion pumps typically have several characteristics in common. Namely, they are preferably light-weight, portable, conveniently operable, comfortable and, most importantly, they are operationally accurate and reliable. To achieve these goals, many different methods have been employed for infusing a fluid medicament to a patient. In each case, it is essential that the fluid medicament be somehow accurately and reliably moved from the source of fluid medicament to the patient.

Typically, many infusion pumps function by generating a mechanical pressure on fluid medicament at its source. Also, using a different functionality, peristaltic pumps operate by direct engagement with an elongated elastomeric fluid channel, and imposing axially directed forces against the fluid as it moves through the infusion tube. Various combinations of these functionalities are also possible.

Apart from the traditional methods for moving a fluid through a tube, the present invention recognizes that the reactionary forces acting within a resilient elastomeric fluid channel as it relaxes and transitions from a stressed configuration back to an unstressed configuration can also be beneficially employed to assist fluid flow. Further, the present invention also recognizes that properly employed sub-ambient pressures on fluid medicament in a collapsible bag can also be beneficial for this same purpose. Moreover, the present invention recognizes that the combined efforts of elastomeric reflex and sub-ambient over-pressures can allow for an effective pumping action for an insulin infusion pump.

With the above in mind, it is an object of the present invention to provide a portable infusion pump that provides sub-ambient over-pressures on a collapsible fluid medicament bag for its operation without relying on a mechanical pump. Another object of the present invention is to provide a portable infusion pump which employs the radial effect of elastomeric reflex from an infusion tube as a primary means for its pumping function. Still another object of the present invention is to provide an infusion pump that, in combination, relies on sub-ambient pressures and elastomeric reflex forces for its pumping function. Yet another object of the present invention is to provide a portable infusion pump that is easy to use, simple to manufacture and relatively cost effective.

### SUMMARY OF THE INVENTION

In accordance with the present invention a portable pump is provided for infusing a fluid medicament to a patient from a replaceable infusion unit. As envisioned for the present invention, the infusion unit will include a collapsible bag which holds the fluid medicament, a cannula needle set which establishes fluid communication with the patient, and an elastomeric fluid channel which interconnects the collapsible bag in fluid communication with the cannula needle set.

Structurally, the portable pump requires a pressure shell which is adapted to create an airtight pressure chamber. The pressure chamber will have a volume V_{c} which is sufficient for holding the collapsible bag, while the elastomeric fluid channel extends from the collapsible bag, and further therefrom outside the pressure shell. A pressure sensor is mounted on the pressure shell to monitor chamber pressure, P_{c}, within the pressure chamber, and an equilibration valve is mounted on the pressure shell to periodically equilibrate the chamber pressure P_{c} with the ambient air pressure P_{amb}.

The fluid channel is made of an elastomeric material having a predetermined modulus of elasticity, λₑ. Structurally, the fluid channel has a first end that is connected in fluid communication with the collapsible bag. The fluid channel also defines an operational segment that extends in a distal direction from the first end toward a second end where it connects with the cannula needle set. Between its first and second ends, the fluid channel is operationally engaged with a pinch/squeeze mechanism.

Operationally, the pinch/squeeze mechanism is engaged with the elastomeric fluid channel for the dual purpose of cyclically decreasing and increasing a predetermined infusion volume, Vᵢ, in the fluid channel. In this operation, when the pinch/squeeze mechanism moves to occlude the elastomeric channel, an infusion volume Vᵢ of fluid medicament is pushed through the lumen of the channel. Also, as the channel is being occluded, the elastic material of the channel in the location of the occlusion is stressed. With this action an infusion volume Vᵢ is displaced and infused to the patient. On the other hand, when the pinch/squeeze mechanism is withdrawn to open and dilate, the elastic material of the channel becomes unstressed. It is an important aspect of the present invention that as the elastic material of the channel becomes unstressed, an infusion volume Vᵢ is drawn from the collapsible bag and into the location in the channel for subsequent engagement with the pinch/squeeze mechanism.

A controller is connected in combination with the pressure sensor, the equilibration valve and the pinch/squeeze mechanism. Specifically, the controller is used to control and coordinate the infusion of a volume Vᵢ of fluid medicament to the patient. As noted above, this operation is done primarily with the pinch/squeeze mechanism by imposing and then relieving radially directed elastomeric stresses on the fluid channel. It happens that as a volume Vᵢ is removed from the chamber, P_{c} will incrementally decrease. Thus, for the present invention it is necessary to periodically equilibrate P_{c} with the ambient pressure, P_{amb}.

Specifically, equilibration of the pressure chamber is accomplished in accordance with the predetermined pressure profile which establishes acceptable ranges and values for the decreasing changes in P_{c}. These changes are detected by the pressure sensor and monitored by the controller. Moreover, it is to be appreciated that changes in P_{c} correspond to volume changes Vᵢ of the collapsible bag within the pressure chamber.

In detail, the pressure profile establishes acceptable operating pressure ranges for P_{c} during each duty cycle Δt of the pinch/squeeze mechanism. The controller thereby monitors a pressure change ΔP_{c} for each duty cycle (ΔP_{c}/Δt). It also identifies an infusion volume Vᵢ of fluid medicament that has been infused to the patient during each duty cycle. As indicated above, the controller activates the equilibration valve to equilibrate P_{c} in the pressure chamber with the ambient pressure P_{amb} for the next duty cycle whenever there is a total pressure drop to a minimum pressure Pₘᵢₙ in the pressure chamber.

Structurally, the pinch/squeeze mechanism comprises, in combination, a base member, a piston unit, and a motorized cam shaft. In this combination, the base member is formed with an elongated U-shaped groove for receiving a portion of the fluid channel's operational segment. The groove has a first side which is formed as a platen and a second side, which is parallel to the first side, where the piston unit is located. Included with the piston unit is an upstream valve, a drive piston, and a downstream valve which are aligned in order with each other in the distal direction along the operational segment of the enclosed fluid channel positioned in the groove. The rotatable, motorized cam shaft is mounted on the base member and connected with the controller for activating the piston unit to maintain Vᵢ constant during the duty cycle in accordance with the pressure profile.

For a detailed understanding of an operation of the present invention, consider that during each duty cycle, the present invention relies on concerted work from both an elastomeric expansion of the fluid channel, Uₑ, and a sub-ambient pressurized collapse of the fluid medicament bag, Uₚ. Together, the total work, Uₜₒₜₐₗ=Uₑ+Uₚ, must always be greater than the minimum level of work, Uₘᵢₙ, that is required to move an infusion volume Vᵢ of fluid medicament from the collapsible bag and into the fluid channel (i.e., Uₜₒₜₐₗ=Uₑ+Uₚ>Uₘᵢₙ). Operationally, this concerted work must be accomplished within a predetermined time interval Δt. The import here is that, with the limitation Uₘᵢₙ in mind, the respective forces for doing the work Uₑ and Uₚ will predictably diminish with time.

In this context, an infusion volume, Vᵢ, must be infused to a patient during the predetermined time interval Δt. Thus, the infusion rate Vᵢ/Δt, must be maintained to ensure that Uₑ+Uₚ>Uₘᵢₙ. Stated differently, the infusion rate Vᵢ/Δt needs to be continuously satisfied by the combined effects of an elastomeric expansion of the fluid channel, and the collapse of the fluid bag in the pressurized chamber. An operational analysis of this relationship is best appreciated by separate considerations of Uₑ and Uₚ.

From a materials perspective, work done by the expanding elastomeric channel is a function of the modulus of elasticity λₑ of the elastomeric material that is used to manufacture the channel. In the event, as the elastomeric material rebounds from a stressed condition, to thereby open the fluid channel at the location where it was squeezed, it will do the work Uₑ. By analogy, Uₑ can be considered as the action of a radial force, Fᵣ, acting through a distance d, in a direction perpendicular to fluid flow, at the location on the elastomeric fluid channel where it was squeezed. As noted above, Fᵣ is generated by internal forces dependent on λₑ of the material.

The overall consequence from the diminishing values of Fᵣ and P_{c} is that both Uₑ and Uₚ diminish over time, albeit at different rates. In the case of Uₚ, however, P_{c} can be periodically equilibrated to P_{amb} on a short-term basis. In contrast, Uₑ has no such short-term reenergizing capability. Nevertheless, an efficient operation is possible as long as Uₜₒₜₐₗ=Uₑ+Uₚ>Uₘᵢₙ is satisfied and Vᵢ/Δt can be maintained.

As a supplementary feature of the present invention, an operation of the system involves controlling sub-ambient pressures p_{c} in the airtight chamber of the pressure shell in an operation that is synchronized with an external fluid control unit. Structurally, the fluid control unit includes a motor-driven camshaft that is rotated at an angular velocity ω to establish a system duty cycle t_{ω} with each 360° rotation of the shaft. For pressure control, the system also uses a pressure sensor to monitor p_{c}, and it includes an equilibration valve for reestablishing ambient pressure p_{amb} in the airtight chamber of the pressure shell at an appropriate time in the duty cycle. Like before, for this synchronized embodiment of the system, p_{c} will follow a predetermined pressure profile.

As disclosed above, a pressure shell is provided for holding a collapsible bag in its airtight chamber under sub-ambient pressures, p_{c}. Also, an elastomeric fluid channel which is connected in fluid communication with the collapsible bag extends outwardly from the pressure shell. For purposes of the present invention, the fluid control unit is engaged with the elastomeric fluid channel at a location outside the pressure shell.

For its engagement with the elastomeric fluid channel, the fluid control unit includes an upstream valve, a piston/platen combination, and a downstream valve. These components are all sequentially aligned along the camshaft to cyclically manipulate the elastomeric fluid channel through a plurality of configurations, in a predetermined sequence, during each duty cycle. The functionality of these configurations is best understood by considering that the pressure profile to be followed by sub-ambient pressures, p_{c}, has two distinct phases during each duty cycle. These are a “dispense phase” and a “draw phase”.

During the dispense phase an infusion volume, Vᵢ, of liquid medicament is dispensed by the system. For this operation, the upstream valve of the fluid control unit is closed, and the downstream valve is open. The piston of the fluid control unit is then advanced laterally against the elastomeric fluid channel to create an over pressure pₒ in the elastomeric fluid channel to dispense liquid medicament. It is an important operational feature of the present invention that the equilibration valve is activated during the dispense phase of the duty cycle to reset an ambient pressure p_{amb} in the airtight chamber.

During the draw phase, an infusion volume, Vᵢ, of liquid medicament is withdrawn from the collapsible bag and repositioned in the elastomeric fluid channel where it will be dispensed during the next duty cycle. For this operation, the upstream valve of the fluid control unit is open, and the downstream valve is closed. The piston is then withdrawn from the elastomeric fluid channel to thereby allow the elastomeric fluid channel to unstress and expand to create an under pressure pᵤ in the elastomeric fluid channel that will draw liquid medicament from the collapsible bag and into the elastomeric fluid channel. In accordance with the present invention, the elastomeric qualities of the fluid channel are such that resilient forces are established within the material when it is stressed. Specifically, this occurs as the elastomeric fluid channel is collapsed by the piston during the dispense phase. Subsequently, it is when the piston is withdrawn from the elastomeric fluid channel in the draw phase that the action of resilient forces in the elastomeric fluid channel creates the pressure pᵤ.

Overall control of the system is provided by a controller. In combination, the controller is connected to the fluid control unit which, in turn, is engaged with the elastomeric fluid channel. The controller is also connected to the pressure sensor and to the equilibration valve, both of which are mounted on the pressure shell. Moreover, the controller is preprogrammed with the pressure profile to cyclically configure the fluid control unit for its operations during the dispense phase and the draw phase as described above. In these operations, pₒ>p_{c}>pᵤ.

In a preferred embodiment of the present invention, the motor-driven camshaft is interconnected between the controller and the equilibration valve. In this configuration, the equilibration valve is engaged directly with the camshaft. Thus, the equilibration valve is mechanically synchronized with the rotation of the camshaft during the dispense phase. In a different embodiment, the controller is interconnected between the motor and the equilibration valve. With this alternate embodiment the controller operates the equilibration valve electronically with impulses from the controller that correspond with the rotation of the motor's camshaft during the dispense phase.

Additional features for the synchronized version of the present invention include the fact that the time duration t_{ω} of the duty cycle is dependent on the rotational velocity ω of the camshaft. Also to be considered is a safety feature based on the fact that when Δp_{c}/Δt does not comply with the pressure profile, during the draw phase of the duty cycle, an occlusion is indicated requiring a stop to the operation of the system.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is an interactive layout of operative components for the present invention and their cooperative interactions;

FIG. 2 is a pressure profile in accordance with the present invention;

FIGS. 3A-D show sequential configurations of the piston unit of a pinch/squeeze mechanism during a duty cycle in accordance with the present invention, wherein FIG. 3A is a pre-fill configuration, FIG. 3B is a fill configuration,

FIG. 3C is a pre-dispense configuration, and FIG. 3D is a dispense configuration; and

FIG. 4 is a logic flow chart for the controller operation implementing the pressure profile;

FIG. 5A is a graphical presentation of the predetermined pressure profile shown for the present invention;

FIG. 5B is a graphical presentation of the dispense/draw effect the pressure profile of FIG. 5A has on sequential infusion volumes of liquid medicament as they are manipulated by the present invention during its duty cycle;

FIG. 6A is a schematic presentation of an embodiment of the present invention wherein the equilibration valve of the present invention is operated electronically; and

FIG. 6B is a schematic presentation of an embodiment of the present invention wherein the equilibration valve of the present invention is operated mechanically.

### DESCRIPTION OF THE PREFERRED EMBODIMENTS

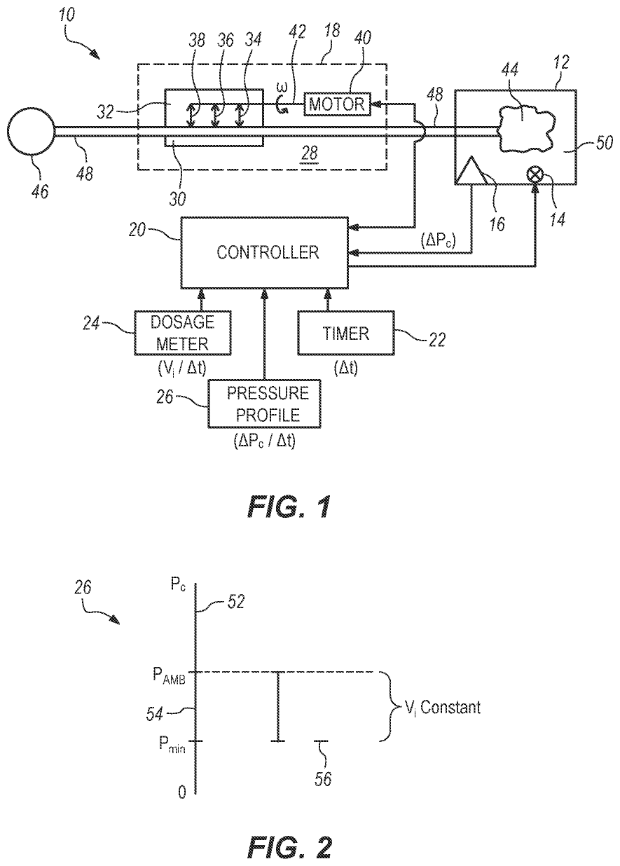

Referring initially to FIG. 1, a schematic diagram of an infusion pump in accordance with the present invention is shown and generally designated 10. As shown, the pump 10 includes a pressure shell 12 on which is mounted an equilibration valve 14 and a pressure sensor 16. Also included with the pump is a pinch/squeeze mechanism 18 and a controller 20. In detail, the controller 20 is connected with the equilibration valve 14 and a pressure sensor 16 on the pressure shell 12. An airtight pressure chamber 50 that is selectively enclosed within the pressure shell 12. The controller 20 is also electronically connected with the pinch/squeeze mechanism 18.

FIG. 1 further shows that the controller 20 includes a timer 22 for monitoring, establishing, and controlling a duty cycle Δt for the pump 10. The controller 20 also includes a dosage meter 24 which is set by a user/patient (not shown) to establish the appropriate infusion volume Vᵢ that is be infused to the user during each duty cycle (Vᵢ/Δt). A pressure profile 26, is included with the controller 20 which provides detailed instructions insofar as an operation of the pump 10 is concerned for compliance with a clinically provided, predetermined operational protocol.

Still referring to FIG. 1, the pinch/squeeze mechanism 18 is shown to include a base member 28 having a platen 30 and an opposed piston unit 32 mounted thereon. In detail, the piston unit 32 includes an upstream valve 34, a drive piston 36, and a downstream valve 38 which are aligned sequentially with each other on the piston unit 32, and across from the platen 30, to form a groove on the base member 28 therebetween. Also mounted on the base member 28 is a motor 40 for rotating a cam shaft 42. In combination, the cam shaft 42 is engaged with the piston unit 32, and the motor 40 is connected with the controller 20 for rotating the cam shaft 42 at an appropriate angular velocity ω. As intended for the present invention, the angular velocity ω is established by the controller 20 to establish the desired infusion volume Vᵢ of fluid medicament during each duty cycle Δt.

As intended for the present invention, the pump 10 is designed for an operational engagement with a disposable infusion unit that includes: a collapsible bag 44 for holding the fluid medicament that is to be infused to the patient, a cannula needle set 46, and an elastomeric fluid channel 48 that is connected to establish fluid communication between the collapsible bag 44 and the cannula needle set 46. Structurally, the collapsible bag 44 is dimensioned to be received into a pressure chamber 50 that is created when the pressure shell 12 is closed.

Several important structural/functional characteristics of the pressure chamber 50 must be satisfied when the pressure chamber 50 is closed. For one, the pressure chamber 50 must be airtight when the pressure shell 12 is closed. For another, the air volume of the pressure chamber 50 inside a closed pressure shell 12 must be greater than the volume of the collapsible bag 44, when the collapsible bag 44 is filled to its full capacity. This is done to optimize the efficacy of an external sub-ambient pressure in the pressure chamber 50 against the collapsible bag 44 during an operation of the pump 10. Further, the chamber pressure P_{c}, as measured by the pressure sensor 16 inside the pressure chamber 50, must be continuously monitored by the controller 20 during an operation of the pump 10.

With consideration of the elastomeric fluid channel 48, it is important that the fluid channel 48 be made of an elastomeric material which has a modulus of elasticity λₑ that causes a relatively rapid transition (rebound/reset) from a stressed configuration back to an unstressed configuration. Specifically, the elastomeric fluid channel 48 is structurally formed with a lumen to transport fluid medicament from the collapsible bag 44 to the cannula needle set 46. In an operation of the present invention, this requires that a portion of the fluid channel 48 be cyclically stressed (collapsed) and unstressed (dilated) by a radially acting, reciprocating force ±F as the lumen of the fluid channel 48 is mechanically collapsed (+F) by the piston unit 32 and dilated by elastomeric forces (−F) from the fluid channel 48. From an operational perspective, this action causes the elastomeric material of the fluid channel 48 to generate a force (−F) that reopens the lumen of the fluid channel 48, and assists the force P_{c} in the pressure chamber 50 in drawing fluid medicament from the collapsible bag 44 and into the elastomeric fluid channel 48.

A pressure profile in accordance with the present invention is shown in FIG. 2, where it is generally designated 26. In FIG. 2 it is shown that the pressure profile 26 includes a line graph 52 of pressures P_{c} for the pressure chamber 50. Specifically, line graph 52 indicates that the pressure profile 26 has an operating range 54 that extends between an ambient pressure P_{amb} and a minimum pressure Pₘᵢₙ. Importantly, FIG. 2 shows the relationship between the operating range 54 of the pump 10 and the ambient pressure P_{amb} of the environment where the pump 10 is to be operated. Moreover, FIG. 2 indicates that P_{c} is to be monitored and controlled by the controller 20 to maintain a constant infusion volume Vᵢ for the patient during an operation of the pump 10. FIG. 2 also indicates the pressure profile 26 establishes an equilibration point 56, at the pressure Pₘᵢₙ, where P_{c} is equilibrated back to P_{amb} for a continued operation of the pump 10.

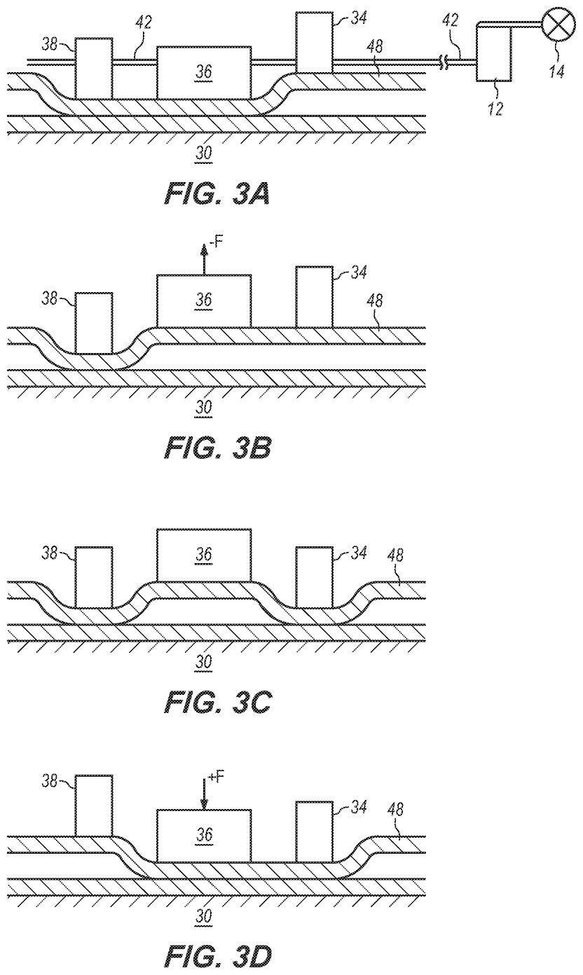

With reference to FIGS. 3A-3D, it is to be appreciated that the pressure profile 26 is based on a duty cycle for the piston unit 32 having a time duration, Δt. Functionally, Δt is the time duration for each sequential 360° rotation of the cam shaft 42. Further, during each Δt, the radially directed interactive forces ±F of the drive piston 36 and the fluid channel 48, respectively, are accomplished during each duty cycle Δt by a sequence of configurations for the piston unit 32.

In FIG. 3A, a pre-fill configuration is shown for the piston unit 32 wherein the downstream valve 38 is closed, the drive piston 36 has been radially advanced to stress the fluid channel 48 against the platen 30, and the upstream valve 34 is open to establish fluid communication between the piston unit 32 and fluid medicament in the collapsible bag 44.

FIG. 3B shows a fill configuration for the piston unit 32 wherein the downstream valve 38 remains closed, while the upstream valve 34 remains open, and the drive piston 36 is radially withdrawn from the platen 30 to unstress the enclosed fluid channel 48 for an elastic rebound with a force −F from its stressed configuration. Thus, fluid medicament is drawn from the collapsible bag 44 by elastomeric rebound of the fluid channel 48 and into the fluid channel 48 as the fluid channel 48 dilates during rebound from its stressed configuration.

FIG. 3C shows a pre-dispense configuration for the piston unit 32 wherein the downstream valve 38 remains closed, the drive piston 36 remains withdrawn from the platen 30 and the upstream valve 34 is closed.

Finally, in FIG. 3D, a dispense configuration for the piston unit 32 is shown wherein the upstream valve 34 remains closed, the downstream valve 38 is opened, and the drive piston 36 is radially advanced with a force +F toward the platen 30 to pump fluid medicament from the piston unit 32 in a distal direction downstream into the operational segment of the fluid channel 48 for infusion to the patient.

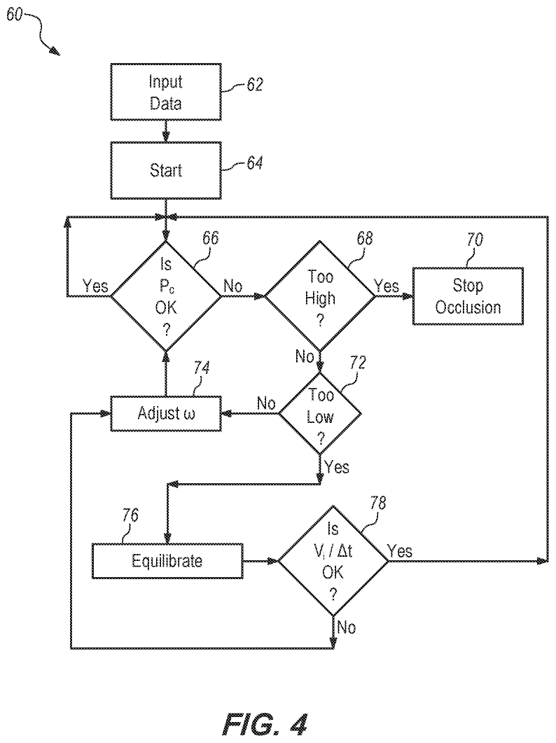

An operation of the pump 10, in accordance with the pressure profile 26, will be best appreciated with reference to the logic flow chart 60 shown in FIG. 4. There it will be seen that the action block 62 requires input data. Specifically, this input data will include the value for Pₘᵢₙ required for the pressure profile 26. The input data will also require values for the fluid medicament infusion volume Vᵢ, and a start value for the duty cycle Δt. With required input, action block 64 indicates that pump 10 can be started.

At the start of an operation of the pump 10, inquiry block 66 determines whether the chamber pressure P_{c} in the pressure chamber 50 is OK. According to inquiry block 66, if the answer is YES, the operation continues. However, if the answer is NO, inquiry block 68 determines whether P_{c} is above P_{amb}. From this inquiry, if P_{c}>P_{amb} an occlusion may be indicated and, in accordance with action block 70, the pump 10 should be stopped.

On the other hand, if P_{c}<P_{amb}, inquiry block 72 determines whether P_{c} is too low. Stated differently, the inquiry block 72 determines whether P_{c} is within the operating range 54 established by the pressure profile 26 (see FIG. 2). If the response from inquiry block 72 is NO, indicating that P_{c} is still within the operating range 54, the action block 74 indicates that, for continued operation, an optional action is to adjust the angular velocity ω of cam shaft 42. It is noted that adjusting w will also result in a change of the duty cycle Δt for pump 10 which, for any number of reasons, may be desirable.

When the response of inquiry block 72 is YES, the action block 76 indicates that the controller 20 will activate the equilibration valve 14 on pressure shell 12. This is done to equilibrate P_{c} in the pressure chamber 50 of pressure shell 12 with the ambient pressure P_{amb}. The next determination for the operation of the pump 10 is indicated by inquiry block 78, where Vᵢ is evaluated in the context of the duty cycle Δt. Specifically, this evaluation begins with P_{c}=P_{amb} when the response of inquiry block 78 is YES, and it continues through subsequent successive duty cycles Δt for as long as inquiry block 72 indicates the pressure profile 26 is satisfied. Thus, it is inquiry block 78 that determines when P_{c} requires equilibration. FIG. 4 also shows that when the response of inquiry block 78 is NO, it may be necessary to adjust ω of motor 40.

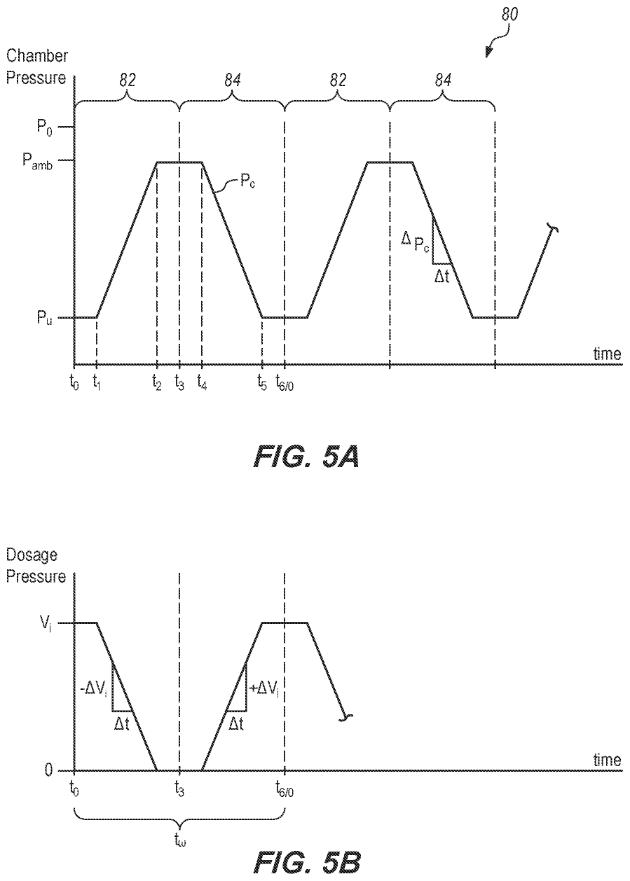

A supplemental feature for the operation of a pump 10 provides for a coordinated synchronization between an operation of the equilibration valve 14, to maintain sub-ambient pressures p_{c} in the pressure chamber 50, and the creation of operational pressures in the elastomeric fluid channel 48 established by the pinch/squeeze mechanism 18. As shown in FIG. 5A, this coordination is to be accomplished in compliance with a pressure profile generally designated 80. Specifically, the pressure profile 80 is established for variations in the sub-ambient pressures p_{c} inside the airtight pressure chamber 50.

As shown, the pressure profile 80 includes both a dispense phase 82 and a draw phase 84. The equilibration valve 14 must not be activated during the draw phase 84. Instead, activation of the equilibration valve 14 must be made sometime during the dispense phase 82. In the event, it is necessary that pump activity during the phases 82 and 84 be coordinated.

In the dispense phase 82 of the pressure profile 80, the pinch/squeeze mechanism 18 creates an operational over pressure pₒ in the elastomeric fluid channel 48 as the drive piston 36 is pushed against the fluid channel 48. Also, during the dispense phase 82, the upstream valve 34 pinches the elastomeric fluid channel 48 to isolate the pressure shell 12 from the elastomeric fluid channel 48. As shown in FIG. 5B, with this configuration of the pinch/squeeze mechanism 18, an infusion volume Vᵢ of liquid medicament is dispensed from the elastomeric fluid channel 48. Simultaneously, as indicated in FIG. 5A, the equilibration valve 14 is activated to cause a pressure rise of p_{c} in the airtight pressure chamber 50 from a minimum Pᵤ back to P_{amb}.

On the other hand, in the draw phase 84 of the pressure profile 80, the pinch/squeeze mechanism 18 is configured with the downstream valve 38 closed and the upstream valve 34 open. Thus, as the drive piston 36 is withdrawn to unstress the resilient elastomeric fluid channel 48, a dosage volume Vᵢ is drawn into the elastomeric fluid channel 48 (see FIG. 5B), with a consequent transfer decrease in volume Vᵢ in the collapsible bag 44. Simultaneously, during the draw phase 84, the equilibration valve 14 has been deactivated to establish an airtight condition in the pressure chamber 50. Consequently, the chamber pressure p_{c} decreases from p_{amb} to the minimum pᵤ during the draw phase 84 as shown in FIG. 5A.

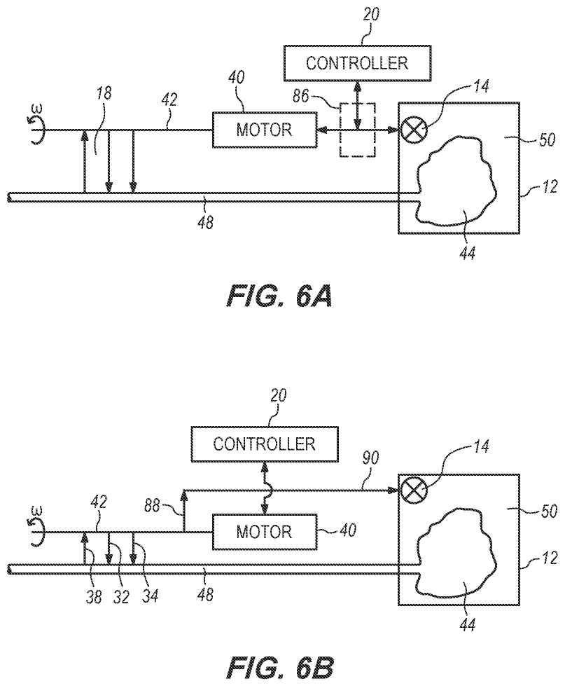

FIGS. 6A and 6B show alternative embodiments for establishing a coordinated synchronization between an operation of the pinch/squeeze mechanism 18 for infusing liquid medicament from the elastomeric fluid channel 48, and an operation of the equilibration valve 14 for controlling the chamber pressure p_{c} in the pressure chamber 50 to introduce liquid medicament into the elastomeric fluid channel 48. One embodiment, FIG. 6A, is electronic, and the other embodiment, FIG. 6B, is mechanical. Both are based on a coordination of the angular rotation w of the cam shaft 42 with the activation of the equilibration valve 14 during the dispense phase 82 of the pressure profile 80.

In FIG. 6A, a circuit timer 86 is interconnected between the motor 40, the controller 20 and the equilibration valve 14. In this combination, the controller 20, which is preprogrammed with the pressure profile 80, connects with the motor 40 via circuit timer 86. Accordingly, the motor 40 provides w information to the controller 20 which, in turn, uses the information to determine an angular orientation of the cam shaft 42. The controller 20 then activates the equilibration valve 14 during the dispense phase 82 in accordance with the requirements of the dispense phase 82 of the pressure profile 80.

In FIG. 6B a valve drive cam 88 is mounted on the cam shaft 42. Further, a connecting link 90 is provided between the drive cam 88 and the equilibration valve 14. In this combination, the motor 40 provides ω information which mechanically determines an angular orientation of the cam shaft 42. The connecting link 90 then activates the equilibration valve 14 during the dispense phase 82 in accordance with the requirements of the dispense phase 82 of the pressure profile 80.

While the particular System for Drawing Fluid From a Bag Under Sub-Ambient Conditions as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for cyclically drawing a liquid medicament from a collapsible bag under sub-ambient pressure conditions in a coordinated synchronization with operational pressures which comprises:
a collapsible bag for holding the liquid medicament, wherein the collapsible bag is connected in fluid communication with an elastomeric fluid channel;
a pressure shell having an airtight chamber for holding the collapsible bag therein with the fluid channel extending outwardly from the pressure shell;
a pressure sensor mounted on the pressure shell for sensing a sub-ambient pressure p_{c} in the airtight chamber;
an equilibration valve mounted on the pressure shell;
a mechanism for creating an operational under pressure pᵤ in the elastomeric fluid channel, wherein pᵤ<p_{c} to draw liquid medicament from the collapsible bag and into the elastomeric fluid channel, and for alternately creating an operational over pressure pₒ in the elastomeric fluid channel, wherein pₒ>pᵤ to dispense liquid medicament from the elastomeric fluid channel; and
a controller connected to the pressure sensor for determining a change of pressure Δp_{c} in the pressure shell during each cycle, wherein the controller is connected to the equilibration valve to reestablish ambient pressure p_{amb} in the pressure chamber with a coordinated synchronization with pₒ during each cycle, and further wherein the controller determines whether a change in Δp_{c}/Δt is compliant with a predetermined pressure profile.

2. The system of claim 1 wherein the mechanism is a pinch/squeeze mechanism comprising:
a fluid control unit including a rotatable camshaft, wherein the fluid control unit includes an upstream valve, a piston and a downstream valve aligned along the camshaft to cyclically manipulate the elastomeric fluid channel through a plurality of configurations, in a predetermined sequence, for creating the under pressure pᵤ in the elastomeric fluid channel during a duty cycle; and
a motor engaged with the camshaft for cyclically rotating the camshaft though 360° during the duty cycle at a controlled angular velocity ω.

3. The system of claim 2 wherein the elastomeric fluid channel is configured to draw liquid medicament from the collapsible bag and into the elastomeric fluid channel when the upstream valve is open, the downstream valve is closed and the piston is being laterally withdrawn from the elastomeric fluid channel, with a concomitant decrease in Δp_{c} in the pressure shell until the end of each duty cycle when the upstream valve is closed and the downstream valve is open to dispense fluid medicament from the elastomeric fluid channel as the piston is laterally advanced against the elastomeric fluid channel.

4. The system of claim 3 wherein the controller is interconnected between the motor and the equilibration valve to synchronously operate the equilibration valve electronically with impulses from the controller corresponding with the rotation of the camshaft by the motor while liquid medicament is being dispensed from the elastomeric fluid channel.

5. The system of claim 3 wherein the motor is interconnected between the controller and the equilibration valve, wherein the equilibration valve is engaged with the camshaft to synchronously operate the equilibration valve mechanically with the rotation of the camshaft by the motor while liquid medicament is being dispensed from the elastomeric fluid channel.

6. The system of claim 1 wherein the predetermined pressure profile is a plurality of successive pressure duty cycles measured in the airtight chamber of the pressure shell, wherein each duty cycle has a same duration Δt and starts with the ambient pressure P_{amb} established by the equilibration valve, the profile then proceeds through the duty cycle with a decreasing value of pressure in p_{c} in the airtight chamber during Δt at a rate Δp_{c}/Δt and ends at a time determined by the controller.

7. The system of claim 6 wherein the end time of the duty cycle is dependent on the rotational velocity ω of the camshaft.

8. The system of claim 6 wherein Δp_{c}/Δt=0 indicates an occlusion when an operation of the system is to be stopped.

9. A system for cyclically drawing a liquid medicament from a collapsible bag under sub-ambient pressure conditions in a coordinated synchronization with operational pressures which comprises:
a pressure sensor for measuring an air pressure p_{c} inside an airtight chamber of a pressure shell while a collapsible bag filled with a liquid medicament is held in the airtight chamber;
an elastomeric fluid channel extending outwardly from the pressure shell, wherein the elastomeric fluid channel is connected in fluid communication with the collapsible bag inside the airtight chamber;
a fluid control unit engaged with the elastomeric fluid channel outside the pressure shell, wherein the fluid control unit includes a rotatable camshaft on which an upstream valve, a piston and a downstream valve are aligned to cyclically manipulate the elastomeric fluid channel through a plurality of configurations, in a predetermined sequence to create a predetermined pressure profile in the airtight chamber during a duty cycle;
a motor engaged with the camshaft for cyclically rotating the camshaft though 360° during the duty cycle at a controlled angular velocity ω to create an operational under pressure pᵤ in the elastomeric fluid channel, wherein pᵤ is less than p_{c} (pᵤ<p_{c}) to draw liquid medicament from the collapsible bag during a draw phase of the duty cycle and thereafter dispense the liquid medicament from the elastomeric fluid channel with an operational over pressure pₒ during a dispense phase of the duty cycle; and
an equilibration valve to reestablish ambient pressure p_{amb} in the pressure shell during the dispense phase of the duty cycle.

10. The system of claim 9 further comprising a controller connected to the pressure sensor for determining a change of pressure Δp_{c} in the pressure shell during each duty cycle, wherein the controller is connected to the equilibration valve to reestablish ambient pressure p_{amb} in the pressure shell during each cycle, and further wherein the controller determines whether a change in Δp_{c}/Δt is compliant with a predetermined pressure profile.

11. The system of claim 10 wherein the elastomeric fluid channel is configured to draw liquid medicament from the collapsible bag and into the elastomeric fluid channel when the upstream valve is open, the downstream valve is closed and the piston is being laterally withdrawn from the elastomeric fluid channel, with a concomitant decrease in Δp_{c} in the pressure shell until the end of each duty cycle when the upstream valve is closed and the downstream valve is open to dispense fluid medicament from the elastomeric fluid channel as the piston is laterally advanced against the elastomeric fluid channel.

12. The system of claim 11 wherein the controller is interconnected between the motor and the equilibration valve to synchronously operate the equilibration valve electronically with impulses from the controller corresponding with the rotation of the camshaft by the motor while liquid medicament is being dispensed from the elastomeric fluid channel.

13. The system of claim 12 wherein the motor is interconnected between the controller and the equilibration valve, wherein the equilibration valve is engaged with the camshaft to synchronously operate the equilibration valve mechanically with the rotation of the camshaft by the motor while liquid medicament is being dispensed from the elastomeric fluid channel.

14. The system of claim 11 wherein the predetermined pressure profile is a plurality of successive pressure duty cycles measured in the airtight chamber of the pressure shell, wherein each duty cycle has a dispense phase wherein the equilibration valve is activated, and a draw phase wherein the equilibration valve is deactivated.

15. The system of claim 14 wherein the time duration of the duty cycle is dependent on the rotational velocity ω of the camshaft, and when Δp_{c}/Δt does not comply with the pressure profile during the draw phase of the duty cycle, an occlusion is indicated requiring a stop to the operation of the system.

16. A system for following a pressure profile to cyclically draw a liquid medicament from a collapsible bag under sub-ambient pressure conditions in a coordinated synchronization with operational pressures which comprises:
a pressure shell having an airtight chamber for holding the collapsible bag therein under a sub-ambient pressure, p_{c}, with an elastomeric fluid channel in fluid communication with the collapsible bag extending outwardly from the pressure shell, and wherein a pressure sensor and an equilibration valve mounted on the pressure shell;
a fluid control unit, wherein the fluid control unit is engaged with the elastomeric fluid channel and includes an upstream valve, a piston and a downstream valve aligned along the camshaft to cyclically manipulate the elastomeric fluid channel through a plurality of configurations, in a predetermined sequence, to create an operational over pressure pₒ in the elastomeric fluid channel during a dispense phase of a duty cycle, and an operational under pressure pᵤ in the elastomeric fluid channel during a draw phase of the duty cycle, wherein pₒ>p_{c}>pᵤ;
a controller connected to the fluid control unit engaged with the elastomeric fluid channel, and to the pressure sensor and the equilibration valve mounted on the pressure shell, wherein the controller is preprogrammed with the pressure profile to configure the fluid control unit with a closed upstream valve and an open downstream valve during the dispense phase of the duty cycle as the piston is advanced against the elastomeric fluid channel to create pₒ therein to dispense liquid medicament therefrom, and to configure the fluid control unit with an open upstream valve and a closed downstream valve during the draw phase of the duty cycle as the piston is withdrawn from the elastomeric fluid channel to create pᵤ in the elastomeric fluid channel to draw liquid medicament from the collapsible bag and into the elastomeric fluid channel, and further wherein the equilibration valve is activated during the dispense phase of the duty cycle to reset an ambient pressure p_{amb} in the airtight chamber.

17. The system of claim 16 further comprising a motor with a rotatable shaft, wherein the shaft is engaged with the fluid control unit and is rotated at an angular velocity ω to establish a system duty cycle with a rotation of the shaft through 360°.

18. The system of claim 17 wherein the controller is interconnected between the motor and the equilibration valve to synchronously operate the equilibration valve electronically with impulses from the controller corresponding with the rotation of the camshaft by the motor while liquid medicament is being dispensed from the elastomeric fluid channel.

19. The system of claim 17 wherein the motor is interconnected between the controller and the equilibration valve, wherein the equilibration valve is engaged with the camshaft to synchronously operate the equilibration valve mechanically with the rotation of the camshaft by the motor while liquid medicament is being dispensed from the elastomeric fluid channel.

20. The system of claim 17 wherein the time duration of the duty cycle is dependent on the rotational velocity ω of the camshaft, and when Δp_{c}/Δt does not comply with the pressure profile during the draw phase of the duty cycle, an occlusion is indicated requiring a stop to the operation of the system.
